(19) United States
(12) Patent Application Publication
(10) Pub. No.: US 2015/0201908 A1
(43) Pub. Date: Jul. 23, 2015

(54) METHOD AND SYSTEM FOR ADAPTIVE BLOOD FLOW VISUALIZATION BASED ON REGIONAL FLOW CHARACTERISTICS

(71) Applicant: General Electric Company, Schenectady, NY (US)

(72) Inventors: Eunji Kang, Wauwatosa, WI (US); Menachem Halmann, Wauwatosa, WI (US)

(73) Assignee: General Electric Company, Schenectady, NY (US)

(21) Appl. No.: 14/161,992

(22) Filed: Jan. 23, 2014

(51) Int. Cl. A61B 8/08; A61B 5/026

(52) U.S. Cl. CPC A61B 8/5223; A61B 8/48; A61B 5/026

(57) ABSTRACT

Various embodiments include a system and method that provide adaptive blood flow visualization by calculating and applying an enhancement factor to each of the pixels in color flow ultrasound image data based on regional flow characteristics detected within the color flow ultrasound image data. The method can include determining for a pixel of a color flow region of interest image, by a processor of an ultrasound system, a lowest percentage of color pixels in a plurality of elongated enhancement analysis windows positioned in the color flow region of interest image. Each of the plurality of elongated enhancement analysis windows includes a pre-defined spatial relationship to an anchor pixel positioned at the pixel. The method includes computing an enhancement factor for the pixel based on the determined lowest percentage of color pixels. The method includes applying the enhancement factor to the pixel.

### CROSS-REFERENCE TO RELATED APPLICATIONS/INCORPORATION BY REFERENCE

Not Applicable

### FIELD OF THE INVENTION

Certain embodiments of the invention relate to ultrasound imaging. More specifically, certain embodiments of the invention relate to a method and system for adaptive blood flow visualization based on regional flow characteristics detected within color flow ultrasound image data.

### BACKGROUND OF THE INVENTION

Ultrasound imaging is a medical imaging technique for imaging organs and soft tissues in a human body. Ultrasound imaging uses real time, non-invasive high frequency sound waves to produce a two-dimensional (2D) image and/or a three-dimensional (3D) image.

Ultrasonic scanners for detecting blood flow are well known. Such systems operate by actuating an ultrasonic transducer array to transmit ultrasonic waves into the object and receiving ultrasonic echoes backscattered from the object. In the measurement of blood flow characteristics, returning ultrasonic waves are compared to a frequency reference to determine the frequency shift imparted to the returning waves by flowing scatterers such as blood cells. This frequency, i.e., phase shift, translates into the velocity of the blood flow. The blood velocity is calculated by measuring the phase shift from firing to firing at a specific range gate.

The change or shift in backscattered frequency increases when blood flows toward the transducer and decreases when blood flows away from the transducer. Color flow images are produced by superimposing a color image of the velocity of moving material, such as blood, over a grayscale anatomical B-mode image. Typically, color flow mode displays hundreds of adjacent sample volumes simultaneously, all laid over a B-mode image and color-coded to represent each sample volume's velocity.

Typically, color flow processors estimate blood flow velocity, blood flow power Doppler imaging (PDI), and blood flow variance. Typically, color flow data is used to modify the color of a region of interest on a display screen. For example, in a typical image, sections with fast moving flows may be displayed with brighter colors and sections with slow moving flows may be displayed with darker colors.

One problem with displaying and interpreting color flow data is that small vessels usually contain slow moving flows and are difficult to detect in ultrasound images. In many ultrasound exams, increased sensitivity is desirable to detect diseases or abnormality earlier. Often times, the measured flow of small vessels in the ultrasound image is overlooked because its color does not stand out from the background image due to its low intensity value. However, when either the flow gain is increased to see these small vessels better or the color map is adjusted to map lower values to a brighter color, the larger flow becomes oversaturated and/or the overall noise level is increased.

Further limitations and disadvantages of conventional and traditional approaches will become apparent to one of skill in the art, through comparison of such systems with some aspects of the present invention as set forth in the remainder of the present application with reference to the drawings.

### BRIEF SUMMARY OF THE INVENTION

A system and/or method is provided for adaptive blood flow visualization based on regional flow characteristics, substantially as shown in and/or described in connection with at least one of the figures, as set forth more completely in the claims.

These and other advantages, aspects and novel features of the present invention, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to provide adaptive blood flow visualization based on regional flow characteristics, in accordance with an embodiment of the invention.

FIG. 2 illustrates exemplary elongated enhancement analysis windows in a region of interest comprising blood vessels, in accordance with an embodiment of the invention.

FIG. 3 is a flow chart illustrating exemplary steps that may be utilized for providing adaptive blood flow visualization based on regional flow characteristics, in accordance with an embodiment of the invention.

FIG. 4 is a graph illustrating an exemplary function of a percentage of color pixels detected in an elongated enhancement analysis window, in accordance with an embodiment of the invention.

### DETAILED DESCRIPTION OF THE INVENTION

Certain embodiments of the invention may be found in a method and system for providing adaptive blood flow visualization by calculating and applying an enhancement factor to each of the pixels in color flow ultrasound image data based on regional flow characteristics detected within the color flow ultrasound image data.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments of the present invention. The following detailed description is, therefore, not to be taken in a limiting sense, and the scope of the present invention is defined by the appended claims and their equivalents.

As used herein, an element or step recited in the singular and proceeded with the word “a” or “an” should be understood as not excluding plural of said elements or steps, unless such exclusion is explicitly stated. Furthermore, references to “an embodiment,” “one embodiment,” “a representative embodiment,” “an exemplary embodiment,” “various embodiments,” “certain embodiments,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

In addition, as used herein, the phrase “pixel” also includes embodiments of the present invention where the data is represented by a “voxel”. Thus, both the terms “pixel” and “voxel” may be used interchangeably throughout this document.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode, CF-mode and/or sub-modes of CF such as TVI, Angio, B-flow, BMI, BMI_Angio, and in some cases also MM, CM, PW, TVD, CW where the “image” and/or “plane” includes a single beam or multiple beams.

Furthermore, the term processor or processing unit, as used herein, refers to any type of processing unit that can carry out the required calculations needed for the invention, such as single or multi-core: CPU, Graphics Board, DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

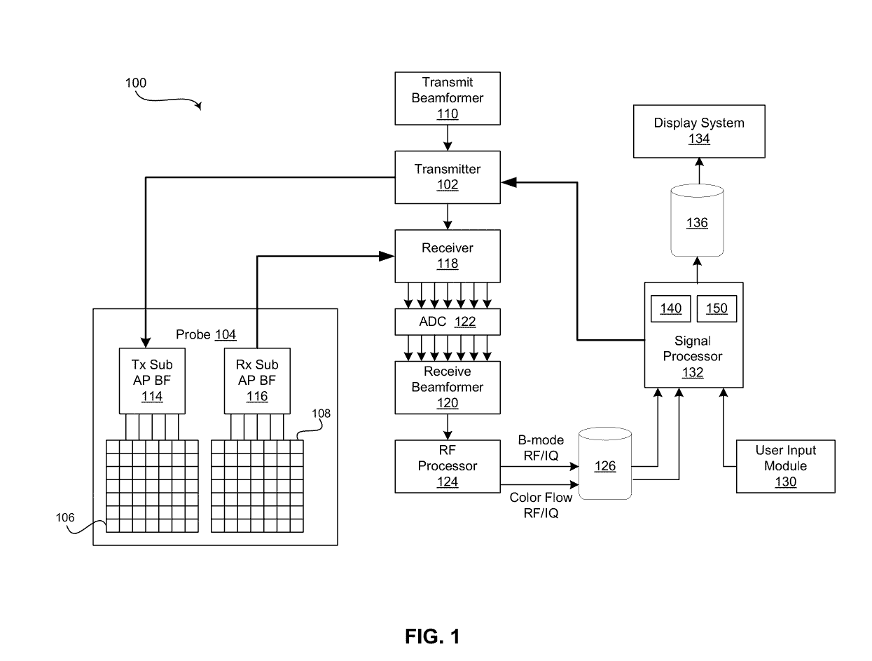

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system that is operable to provide adaptive blood flow visualization based on regional flow characteristics, in accordance with an embodiment of the invention. Referring to FIG. 1, there is shown an ultrasound system 100. The ultrasound system 100 comprises a transmitter 102, an ultrasound probe 104, a transmit beamformer 110, a receiver 118, a receive beamformer 120, a RF processor 124, a RF/IQ buffer 126, a user input module 130, a signal processor 132, an image buffer 136, and a display system 134.

The transmitter 102 may comprise suitable logic, circuitry, interfaces and/or code that may be operable to drive an ultrasound probe 104 to provide, for example, color flow and gray scale imaging. The ultrasound probe 104 may comprise a two dimensional (2D) or three dimensional (3D) array of piezoelectric elements. The ultrasound probe 104 may comprise a group of transmit transducer elements 106 and a group of receive transducer elements 108, that normally constitute the same elements.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The group of transmit transducer elements 106 can be activated to transmit pulse sequences comprising tone burst of length P that are fired repeatedly at a pulse repetition frequency (PRF), which is typically in the kilohertz range. The pulse sequences, including burst lengths P, are different for color flow and B-mode processing. For color flow imaging, P may be 4 to 8 cycles, and the tone burst are focused at the same transmit focal position with the same transmit characteristics. A series of color flow transmit firings focused at the same transmit focal position are referred to as a “packet”. The transmitted ultrasonic signals may be backscattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118.

The receiver 118 may comprise suitable logic, circuitry, interfaces and/or code that may be operable to receive and demodulate the signals from the receive sub-aperture beamformer 116. The demodulated analog signals may be communicated to one or more of the plurality of A/D converters 122.

The plurality of A/D converters 122 may comprise suitable logic, circuitry, interfaces and/or code that may be operable to convert the demodulated analog signals from the receiver 118 to corresponding digital signals. The plurality of A/D converters 122 are disposed between the receiver 118 and the receive beamformer 120. Notwithstanding, the invention is not limited in this regard. Accordingly, in some embodiments of the invention, the plurality of A/D converters 122 may be integrated within the receiver 118.

The receive beamformer 120 may comprise suitable logic, circuitry, interfaces and/or code that may be operable to perform digital beamforming processing to, for example, sum the delayed channel signals received from the plurality of A/D converters 122 and output a beam summed signal. The resulting processed information may be converted back to corresponding RF signals. The corresponding output RF signals that are output from the receive beamformer 120 may be communicated to the RF processor 124. In accordance with some embodiments of the invention, the receiver 118, the plurality of A/D converters 122, and the beamformer 120 may be integrated into a single beamformer, which may be digital.

The RF processor 124 may comprise suitable logic, circuitry, interfaces and/or code that may be operable to demodulate the RF signals. In accordance with an embodiment of the invention, the RF processor 124 may comprise a complex demodulator (not shown) that is operable to demodulate the RF signals to form B-mode I/Q data pairs and color flow I/Q data pairs that are representative of the corresponding echo signals. The B-mode and color flow RF or I/Q signal data may then be communicated to an RF/IQ buffer 126.

The RF/IQ buffer 126 may comprise suitable logic, circuitry, interfaces and/or code that may be operable to provide temporary storage of the B-mode and color flow RF or I/Q signal data, which is generated by the RF processor 124.

The user input module 130 may be utilized to input patient data, surgical instrument data, scan parameters, settings, configuration parameters, change scan mode, and the like. In an exemplary embodiment of the invention, the user input module 130 may be operable to configure, manage and/or control operation of one or more components and/or modules in the ultrasound system 100. In this regard, the user input module 130 may be operable to configure, manage and/or control operation of transmitter 102, the ultrasound probe 104, the transmit beamformer 110, the receiver 118, the receive beamformer 120, the RF processor 124, the RF/IQ buffer 126, the user input module 130, the signal processor 132, the image buffer 136, and/or the display system 134.

The signal processor 132 may comprise suitable logic, circuitry, interfaces and/or code that may be operable to process the ultrasound scan data (i.e., color flow and B-mode RF signal data or IQ data pairs) for generating an ultrasound image for presentation on a display system 134. The signal processor 132 is operable to perform one or more processing operations according to a plurality of selectable ultrasound modalities on the acquired ultrasound scan data. In an exemplary embodiment of the invention, the signal processor 132 may be operable to perform compounding, motion tracking, and/or speckle tracking. Acquired ultrasound scan data may be processed in real-time during a scanning session as the color flow and B-mode echo signals are received. Additionally or alternatively, the ultrasound scan data may be stored temporarily in the RF/IQ buffer 126 during a scanning session and processed in less than real-time in a live or off-line operation.

The ultrasound system 100 may be operable to continuously acquire ultrasound scan data at a frame rate that is suitable for the imaging situation in question. Typical frame rates range from 20-70 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several seconds worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include a grayscale B-mode processing module 140 that comprises suitable logic, circuitry, interfaces and/or code that may be operable to process the B-mode RF signal data or IQ data pairs to form frames provided to the image buffer 136 and/or the display system 134. For example, the grayscale B-mode processing module 140 can form an envelope of the beamsummed receive signal by computing the quantity (I²+Q²)^{1/2}. The envelope can undergo additional B-mode processing, such as logarithmic compression to form the display data. The display data may be converted to X-Y format for video display. The scan-converted frames can be mapped to grayscale for display.

The signal processor 132 may include a color flow processing module 150 that comprises suitable logic, circuitry, interfaces and/or code that may be operable to process color flow RF signal data or IQ data pairs to form frames to overlay on B-mode frames that are provided to the image buffer 136 and/or the display system 134. In a representative embodiment, the color flow processing module provides adaptive blood flow visualization by calculating and applying an enhancement factor to each of the pixels in the color flow ultrasound image data based on regional flow characteristics detected within the color flow ultrasound image data. The color flow processing module 150 may receive a selection from the user input module 130, for example, of a vessel thickness threshold for enhancement. For example, a user may provide an input indicating enhancement for vessels with a thickness of less than 2 millimeters, or any suitable thickness. The vessel thickness threshold can be based on an application, such as an imaging procedure on a particular portion of a patient's anatomy. In various embodiments, the vessel thickness threshold can be a default value.

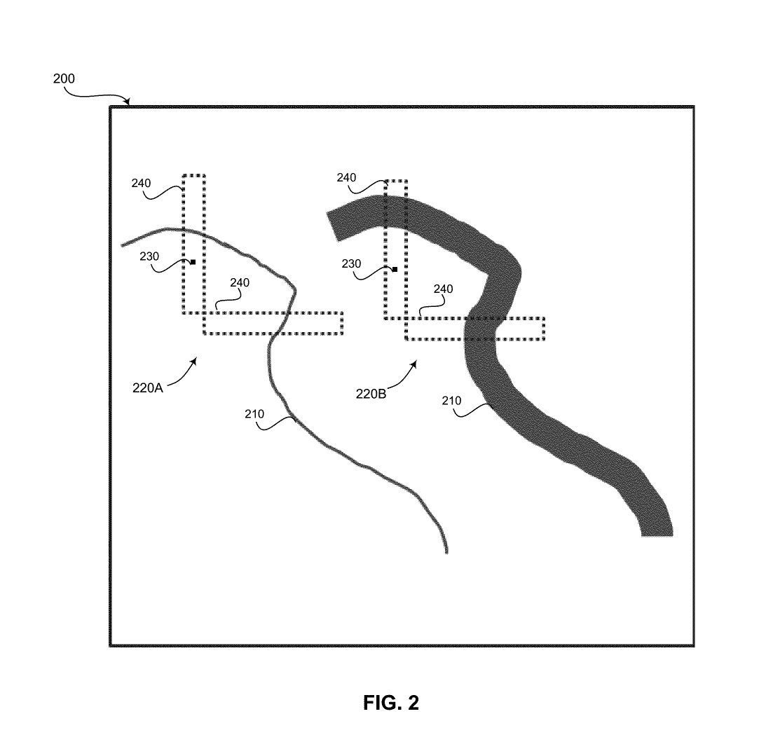

The color flow processing module 150 comprises suitable logic, circuitry, interfaces and/or code that may be operable to determine a size of a plurality of elongated enhancement analysis windows 240 based on the selected vessel thickness threshold and imaging parameters. The imaging parameters can include the number of beams, number of samples, and/or any suitable imaging parameter for determining a resolution and size of a region of interest of the color flow ultrasound image data. The plurality of elongated enhancement analysis windows provide information related to the size of blood vessels 210 that pass through the windows 240 as shown in FIG. 2 and discussed in more detail below. The plurality of elongated enhancement analysis windows 240 has a pre-defined spatial relationship with respect to each other. An anchor pixel 230 is set at a pre-defined position in relation to the plurality of elongated enhancement analysis windows. For example, the anchor pixel 230 may correspond to a pixel in one of the plurality of elongated enhancement analysis windows 240.

The color flow processing module 150 comprises suitable logic, circuitry, interfaces and/or code that may be operable to determine an enhancement factor for each pixel in the region of interest 200 of the color flow ultrasound image data by moving the anchor pixel 230 with the plurality of elongated enhancement analysis windows 240 to each pixel in the region of interest 200 and analyzing the pixels within the plurality of elongated enhancement analysis windows 240. For example, the enhancement factor for each pixel can generally be inversely proportional to a percentage of color pixels in one of the plurality of elongated enhancement analysis windows 240 having the smallest percentage of color pixels. More particularly, the higher percentage of color pixels present in the plurality of elongated enhancement analysis windows 240 when a particular pixel is being analyzed, the more likely that particular pixel belongs to a larger vessel. As such, the color flow processing module 150 provides a higher enhancement factor for pixels corresponding to smaller vessels because those pixels are associated with smaller color pixel percentages determined by analyzing the neighboring pixels within the plurality of elongated enhancement analysis windows.

FIG. 2 illustrates exemplary elongated enhancement analysis windows 240 in a region of interest 200 comprising blood vessels 210, in accordance with an embodiment of the invention. Referring to FIG. 2, a region of interest 200 of color flow ultrasound image data comprises blood vessels 210 and exemplary elongated enhancement analysis windows 240 at a first position 220A and a second position 220B. At each position 220A, 220B, the elongated enhancement analysis windows 240 have a pre-defined spatial relationship with respect to each other and an anchor pixel 230. The elongated enhancement analysis windows 240 can be a first window 240 in a vertical direction and a second window 240 in a horizontal direction, as illustrated in FIG. 2, or any suitable number and arrangement of windows 240. The elongated enhancement analysis windows 240 may be a rectangular shape, oval shape, or any suitable elongated shape. As illustrated in FIG. 2, a higher percentage of color pixels are present in the plurality of elongated enhancement analysis windows 240 at the second position 220B, making it more likely that an anchor pixel 230, if a color pixel, would be associated with a larger vessel. In various embodiments, the anchor pixel 230 with the plurality of elongated enhancement analysis windows 240 is moved to each pixel in the region of interest 200 to determine a percentage of color pixels in the elongated enhancement analysis windows 240 at each position such that each pixel can be associated with an enhancement factor. Although the anchor pixel 230 is shown positioned in the vertical elongated enhancement analysis window, the anchor pixel 230 may be positioned elsewhere in, on, or around any of the plurality of elongated enhancement analysis windows 230 as long as the position of the anchor pixel 230 in relation to the plurality of elongated enhancement analysis windows is consistent throughout the analysis of each of the pixels of the region of interest 200.

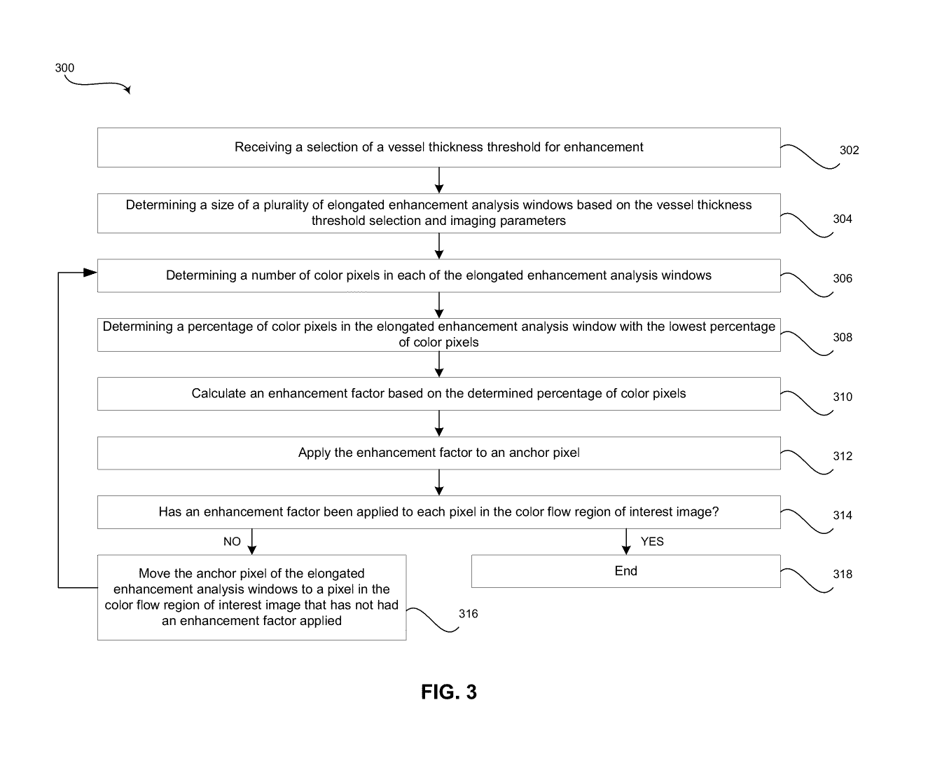

FIG. 3 is a flow chart illustrating exemplary steps that may be utilized for providing adaptive blood flow visualization based on regional flow characteristics, in accordance with an embodiment of the invention. Referring to FIG. 3, there is shown a flow chart 300 comprising exemplary steps 302 through 318. Certain embodiments of the present invention may omit one or more of the steps, and/or perform the steps in a different order than the order listed, and/or combine certain of the steps discussed below. For example, some steps may not be performed in certain embodiments of the present invention. As a further example, certain steps may be performed in a different temporal order, including simultaneously, than listed below.

In step 302, a color flow processing module 150 of a signal processor 132 in the ultrasound system 100 may be operable to receive a selection of a vessel thickness threshold for enhancement. For example, an operator of an ultrasound system may desire to enhance the visualization of vessels with a thickness less than 2 millimeters (mm) based on an application, such as an imaging procedure on a particular portion of a patient's anatomy. The operator can use a user input module 130, for example, to specify the selected vessel thickness threshold for enhancement. Additionally and/or alternatively, the vessel thickness threshold can be a default value.

In step 304, the color flow processing module 150 of the signal processor 132 in the ultrasound system 100 can be operable to determine a size of elongated enhancement analysis windows 240 based on the vessel thickness threshold received at stop 302 and imaging parameters. For example, the color flow processing module 150 automatically translates the vessel thickness threshold into a number of pixels within the elongated enhancement analysis windows 240 based on a number of beams and a number of samples, or any suitable imaging parameters that may be used to determine a resolution and size of a region of interest image 200.

In step 306, the color flow processing module 150 of the signal processor 132 may determine a number of color pixels in each of the elongated enhancement analysis windows 240 at a first position of an anchor pixel 230. The elongated enhancement analysis windows 240, having a pre-defined spatial relationship with respect to each other and an anchor pixel 230, can be a first window 240 in a vertical direction and a second window 240 in a horizontal direction, as illustrated in FIG. 2, or any suitable number and arrangement of windows 240. Referring to the exemplary first position 220A in FIG. 2, the color flow processing module 150 can determine that there are ten (10) color pixels in the first window arranged vertically, and sixteen (16) color pixels in the second window arranged horizontally.

In step 308, the color flow processing module 150 of the signal processor 132 may determine a percentage of color pixels in the elongated enhancement analysis window 240 with the lowest percentage of color pixels. For example, continuing the above example where there are ten (10) color pixels in the first window arranged vertically and sixteen (16) color pixels in the second window arranged horizontally, if each of the elongated enhancement analysis windows 240 has one hundred (100) pixels, the elongated enhancement analysis window 240 with the lowest percentage of color pixels would be the vertical window 240 having ten (10) percent color pixels.

In step 310, the color flow processing module 150 of the signal processor 132 can calculate an enhancement factor for a pixel associated with the current anchor pixel based on the determined percentage of color pixels from step 308. In various embodiments, the enhancement factor for each pixel can generally be inversely proportional to a percentage of color pixels in one of the plurality of elongated enhancement analysis windows 240 having the smallest percentage of color pixels. For example, in an embodiment, the enhancement factor can be calculated as follows:

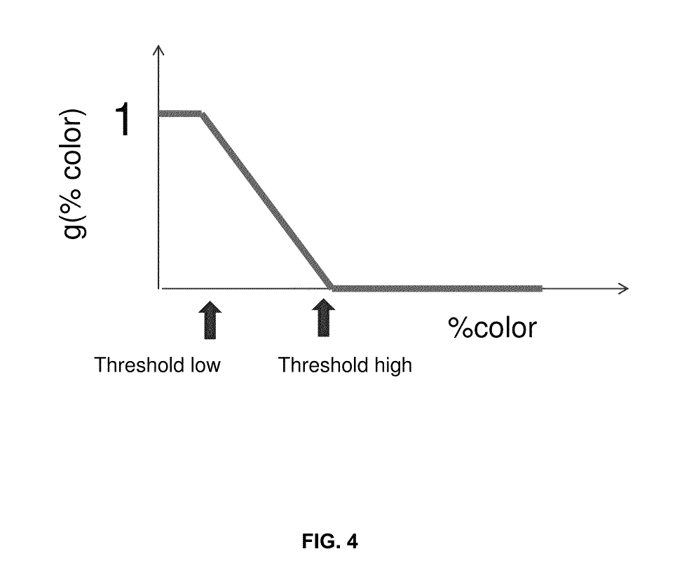

Enhancement Factor=(g(% color)×K)+1

where g(% color) is a function of the percent color and K is a scaling factor. As an example, the function of the percent color g(% color) can be between 0 and 1, where a percentage of color at or below a low color threshold is represented by 1, a percentage of color at or above a high color threshold is represented by 0, and percentages of color between the color thresholds are gradually decreased from 1 to 0 as the percentage of color increases, as illustrated in FIG. 4. The function of percentage color g(% color) may be user-definable and can be linear or non-linear. The scaling factor K can provide a user-adjustable range of enhancement. For example, if K equals 1, the enhancement factor may be between 1 and 2. As another example, if K equals 5, the enhancement factor can be between 1 and 6. Further, if K equals 0.2, the enhancement factor may be between 1 and 1.2.

In step 312, the color flow processing module 150 of the signal processor 132 can apply the enhancement factor to the pixel associated with the anchor pixel. For example, referring to FIG. 2, the enhancement factor applied to the pixel associated with the anchor pixel 230 at a first position 220A would be greater than the enhancement factor applied to the pixel associated with the anchor pixel 230 at a second position 220B because the anchor pixel 230 at the second position is associated with a higher percentage of color pixels in the elongated enhancement analysis window 240 with the lowest percentage of color pixels. The applied enhancement factor has the effect of increasing the brightness of color pixels by the associated factor. Although in various embodiments, each pixel in a region of interest image 200 may be assigned an enhancement factor, the enhancement factor has no effect on the non-color pixels because the non-color pixels do not have a color to enhance.

In step 314, the color flow processing module 150 of the signal processor 132 may determine whether the enhancement factor has been applied to each pixel in the color flow region of interest image 200. In various embodiments, an enhancement factor is determined for each pixel in the region of interest 200 of the color flow ultrasound image data. Alternatively, certain embodiments provide that the enhancement factor can be determined for each color pixel in the region of interest 200.

In step 316, if the enhancement factor has not been applied to each pixel (or each color pixel) in the color flow region of interest image, the color flow processing module 150 of the signal processor 132 can move the anchor pixel 230 with the plurality of elongated enhancement analysis windows 240 to a next non-analyzed pixel (or color pixel) in the region of interest 200 and repeat steps 306 through 314. The color flow processing module 150 of the signal processor 132 repeats steps 306 through 314 for each pixel (or color pixel) of the region of interest 200 such that an enhancement factor is applied to each pixel (or color pixel). In various embodiments, the color flow processing module 150 of the signal processor 132 may calculate the enhancement factor for each pixel (or color pixel) of the region of interest 200 prior to applying the enhancement factors.

In step 318, if the enhancement factor has been applied to each pixel (or each color pixel) in the color flow region of interest image 200, the color flow processing module 150 of the signal processor 132 can apply additional processing and/or output the processed color flow image data to superimpose over the grayscale anatomical B-mode image provided by the grayscale B-mode processing module 140.

FIG. 4 is a graph illustrating an exemplary function of a percentage of color pixels detected in an elongated enhancement analysis window, in accordance with an embodiment of the invention. Referring to FIG. 4, the function of the percent color g(% color) can be between 0 and 1, where a percentage of color at or below a low color threshold is represented by 1, a percentage of color at or above a high color threshold is represented by 0, and percentages of color between the color thresholds are gradually decreased from 1 to 0 as the percentage of color increases. The function of percentage color g(% color), including the high color threshold and low color threshold, may be user-definable. In various embodiments, function of percentage color g(% color) can be linear or non-linear.

Aspects of the present invention have the technical effect of providing adaptive blood flow visualization by calculating and applying an enhancement factor to each of the pixels in color flow ultrasound image data based on regional flow characteristics detected within the color flow ultrasound image data. In accordance with various embodiments of the invention, a method 300 comprises determining 308 for a pixel of a color flow region of interest image 200, by a processor 150, 132 of an ultrasound system 100, a lowest percentage of color pixels in a plurality of elongated enhancement analysis windows 240 positioned in the color flow region of interest image 200. Each of the plurality of elongated enhancement analysis windows 240 comprises a pre-defined spatial relationship to an anchor pixel 230 positioned at the pixel. The method 300 comprises computing 310, by the processor 150, 132, an enhancement factor for the pixel based on the determined lowest percentage of color pixels. The method 300 comprises applying 300, by the processor 150, 132, the enhancement factor to the pixel.

In a representative embodiment, the method 300 comprises receiving 302, by the processor 150, 132, a selection of a vessel thickness threshold for enhancement. In certain embodiments, the method 300 comprises determining 304, by the processor 150, 132, a size of each of the plurality of elongated enhancement analysis windows 240 based on the vessel thickness threshold and imaging parameters. In various embodiments, the imaging parameters comprise a number of beams and a number of samples. In a representative embodiment, the imaging parameters determine a size and a resolution of the color flow region of interest image 200.

In certain embodiments, the determining 308 the lowest percentage of color pixels comprises determining 306 a number of color pixels in each of the plurality of elongated enhancement analysis windows 240. The determining 308 the lowest percentage of color pixels also comprises dividing the number of color pixels in each of the plurality of elongated enhancement analysis windows 240 by a total number of pixels within a respective elongated enhancement analysis window 240. The determining 308 the lowest percentage of color pixels further comprises identifying the lowest percentage of color pixels of the plurality of elongated enhancement analysis windows 240.

In various embodiments, the enhancement factor is computed based on: Enhancement Factor=(g(% color)×K)+1, where g(% color) is a function of the lowest percentage of color pixels and K is a scaling factor. In a representative embodiment, the enhancement factor is generally inversely proportional to the lowest percentage of color pixels. In certain embodiments, the method 300 comprises moving 316 the anchor pixel to a non-analyzed pixel and repeating the determining 308, computing 310, and applying 312 steps if the enhancement factor has not been applied 314 to each pixel in the color flow region of interest 200. In various embodiments, the applying 312 the enhancement factor to the pixel comprises increasing the brightness of a color of the pixel by the enhancement factor.

In accordance with various embodiments of the invention, a system comprises an ultrasound device 100 comprising a processor 150, 132 operable to determine, for a pixel of a color flow region of interest image 200, a lowest percentage of color pixels in a plurality of elongated enhancement analysis windows 240 positioned in the color flow region of interest image 200. Each of the plurality of elongated enhancement analysis windows 240 comprises a pre-defined spatial relationship to an anchor pixel 230 positioned at the pixel. The processor 150, 132 is operable to compute an enhancement factor for the pixel based on the determined lowest percentage of color pixels. The processor 150, 132 is operable to increase the brightness of a color of the pixel by the enhancement factor.

In certain embodiments, the ultrasound device 100 comprises a user input module 130. The processor 150, 132 is operable to receive a selection of a vessel thickness threshold for enhancement from the user input module 130. In a representative embodiment, the processor 150, 132 is operable to determine a size of each of the plurality of elongated enhancement analysis windows based on the vessel thickness threshold and imaging parameters. In various embodiments, the processor 150, 132 is operable to compute the enhancement factor based on: Enhancement Factor=(g(% color)×K)+1, where g(% color) is a function of the lowest percentage of color pixels and K is a scaling factor.

In a representative embodiment, if the enhancement factor has not been applied to each pixel in the color flow region of interest 200, the processor 150, 132 is operable to move the anchor pixel 230 to a non-analyzed pixel. The processor 150, 132 is operable to determine, for the non-analyzed pixel of the color flow region of interest image 200, a lowest percentage of color pixels in the plurality of elongated enhancement analysis windows 240 positioned in the color flow region of interest image 200. Each of the plurality of elongated enhancement analysis windows 240 comprises a pre-defined spatial relationship to the anchor pixel 230 positioned at the non-analyzed pixel. The processor 150, 132 is operable to compute an enhancement factor for the non-analyzed pixel based on the determined lowest percentage of color pixels. The processor 150, 132 is operable to increase the brightness of a color of the non-analyzed pixel by the enhancement factor.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and code (if any is necessary) to perform the function, regardless of whether performance of the function is disabled, or not enabled, by some user-configurable setting.

Other embodiments of the invention may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for providing sequential needle recalibration by correlating a sequence of calibration data for a tracking system to a plurality of corresponding ultrasound probe positions.

Accordingly, the present invention may be realized in hardware, software, or a combination of hardware and software. The present invention may be realized in a centralized fashion in at least one computer system, or in a distributed fashion where different elements are spread across several interconnected computer systems. Any kind of computer system or other apparatus adapted for carrying out the methods described herein is suited. A typical combination of hardware and software may be a general-purpose computer system with a computer program that, when being loaded and executed, controls the computer system such that it carries out the methods described herein.

The present invention may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims.

What is claimed is:

1. A method, comprising:
determining for a pixel of a color flow region of interest image, by a processor of an ultrasound system, a lowest percentage of color pixels in a plurality of elongated enhancement analysis windows positioned in the color flow region of interest image, each of the plurality of elongated enhancement analysis windows having a pre-defined spatial relationship to an anchor pixel positioned at the pixel;
computing, by the processor, an enhancement factor for the pixel based on the determined lowest percentage of color pixels; and
applying, by the processor, the enhancement factor to the pixel.

2. The method according to claim 1, comprising receiving, by the processor, a selection of a vessel thickness threshold for enhancement.

3. The method according to claim 2, comprising determining, by the processor, a size of each of the plurality of elongated enhancement analysis windows based on the vessel thickness threshold and imaging parameters.

4. The method according to claim 3, wherein the imaging parameters comprise a number of beams and a number of samples.

5. The method according to claim 3, wherein the imaging parameters determine a size and a resolution of the color flow region of interest image.

6. The method according to claim 3, wherein the determining the lowest percentage of color pixels comprises:
determining a number of color pixels in each of the plurality of elongated enhancement analysis windows,
dividing the number of color pixels in each of the plurality of elongated enhancement analysis windows by a total number of pixels within a respective elongated enhancement analysis window, and
identifying the lowest percentage of color pixels of the plurality of elongated enhancement analysis windows.

7. The method according to claim 1, wherein the enhancement factor is computed based on:
Enhancement Factor=(g(% color)×K)+1
where g(% color) is a function of the lowest percentage of color pixels and K is a scaling factor.

8. The method according to claim 1, wherein the enhancement factor is generally inversely proportional to the lowest percentage of color pixels.

9. The method according to claim 1, comprising moving the anchor pixel to a non-analyzed pixel and repeating the determining, computing, and applying steps if the enhancement factor has not been applied to each pixel in the color flow region of interest.

10. The method according to claim 1, wherein the applying the enhancement factor to the pixel comprises increasing the brightness of a color of the pixel by the enhancement factor.

11. A system, comprising:
an ultrasound device comprising:
a processor operable to:
determine, for a pixel of a color flow region of interest image, a lowest percentage of color pixels in a plurality of elongated enhancement analysis windows positioned in the color flow region of interest image, each of the plurality of elongated enhancement analysis windows having a pre-defined spatial relationship to an anchor pixel positioned at the pixel;
compute an enhancement factor for the pixel based on the determined lowest percentage of color pixels; and
increase the brightness of a color of the pixel by the enhancement factor.

12. The system according to claim 11, comprising a user input module, wherein the processor is operable to receive a selection of a vessel thickness threshold for enhancement from the user input module.

13. The system according to claim 12, wherein the processor is operable to determine a size of each of the plurality of elongated enhancement analysis windows based on the vessel thickness threshold and imaging parameters.

14. The system according to claim 11, wherein the processor is operable to compute the enhancement factor based on:
Enhancement Factor=(g(% color)×K)+1
where g(% color) is a function of the lowest percentage of color pixels and K is a scaling factor.

15. The system according to claim 11, wherein if the enhancement factor has not been applied to each pixel in the color flow region of interest, the processor is operable to:
move the anchor pixel to a non-analyzed pixel,
determine, for the non-analyzed pixel of the color flow region of interest image, a lowest percentage of color pixels in the plurality of elongated enhancement analysis windows positioned in the color flow region of interest image, each of the plurality of elongated enhancement analysis windows having a pre-defined spatial relationship to the anchor pixel positioned at the non-analyzed pixel;
compute an enhancement factor for the non-analyzed pixel based on the determined lowest percentage of color pixels; and
increase the brightness of a color of the non-analyzed pixel by the enhancement factor.

16. A non-transitory computer readable medium having stored thereon, a computer program having at least one code section, the at least one code section being executable by a machine for causing the machine to perform steps comprising:
determining, for a pixel of a color flow region of interest image, a lowest percentage of color pixels in a plurality of elongated enhancement analysis windows positioned in the color flow region of interest image, each of the plurality of elongated enhancement analysis windows having a pre-defined spatial relationship to an anchor pixel positioned at the pixel;
computing an enhancement factor for the pixel based on the determined lowest percentage of color pixels; and
increasing the brightness of a color of the pixel by the enhancement factor.

17. The non-transitory computer readable medium according to claim 16, comprising receiving a selection of a vessel thickness threshold for enhancement.

18. The non-transitory computer readable medium according to claim 17, comprising determining a size of each of the plurality of elongated enhancement analysis windows based on the vessel thickness threshold and imaging parameters.

19. The non-transitory computer readable medium according to claim 16, wherein the enhancement factor is computed based on:
Enhancement Factor=(g(% color)×K)+1
where g(% color) is a function of the lowest percentage of color pixels and K is a scaling factor.

20. The non-transitory computer readable medium according to claim 16, comprising moving the anchor pixel to a non-analyzed pixel and repeating the determining, computing, and increasing steps if the enhancement factor has not been applied to each pixel in the color flow region of interest.
